SEQUENCE LISTING (2 sequences)

SEQ ID NO: 1 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2005/0244375 A1
(43) Pub. Date: Nov. 3, 2005

(54) Composition and method of cancer treatment

(76) Inventors: Arnold S. Leonard, Minneapolis, MN (US); Daniel A. Saltzman, Mendota Heights, MN (US); Mark J. Mueller, Spooner, WI (US)

Correspondence Address: Richard C. Emery, 4189 Lakewood Avenue, White Bear Lake, MN, 55110-3925, US

(21) Appl. No.: 10/834,587

(22) Filed: Apr. 29, 2004

(51) Int. Cl. C12P 21/04; A61K 48/00; A61K 35/78

(52) U.S. Cl. 424/93.2; 424/732; 424/765

(57) ABSTRACT

The invention discloses a method for treating cancer comprising administering to a patient an effective amount of attenuated Salmonella typhimurium containing a plasmid carrying the coding sequence encoding human interleukin-2 and an oil containing a high antioxidant concentration and a method for administering the composition.

### FIELD OF THE INVENTION

The invention relates to a composition and method of cancer treatment using attenuated salmonella containing the human IL-2 gene combined with highly potent antioxidant oils which have been shown to boost the immune system of the patient.

### BACKGROUND

It is estimated that 150,000 new cases of colorectal cancer occur in North America every year. Of these patients, it is expected that 40 to 50 percent will experience a recurrence within five years. Furthermore, it is known that the 75 to 80 percent of patients with a recurrence have the liver as one of the involved sites for metastasis. Unresectable metastatic carcinoma of the liver continues to have a very poor prognosis despite recent advances with chemotherapeutic and radiotherapeutic strategies, radiofrequency ablation and cryotherapy. It is true that when caught at an early stage, Duke's stages A or B (i.e., malignant invasion confined to the intestinal wall), a multimodal approach of both surgery and chemotherapy have proven to be beneficial. However, when colorectal cancer metastasizes, it usually does so in the liver and if the metastases are unresectable there is currently no effective treatment strategy with a reasonable hope of a cure.

### BRIEF DESCRIPTION OF THE DRAWINGS

FIG. 1 shows the pYA292 plasmid containing the coding sequence encoding the human interleukin-2 protein.

FIG. 2 shows the amino acid sequence (SEQ ID NO: 1) of the human interleukin-2 protein.

FIG. 3 shows the nucleotide sequence (SEQ ID NO:2) encoding the human interleukin-2 protein attached to Salmonella typhimurium χ4550pIL2.

FIG. 4 is a bar graph representing (Tumor Treatment Model) reduced hepatic metastases in response to control (saline), Salmonella typhimurium χ4550 and Salmonella typhimurium χ4550pIL2.

FIG. 5 shows a bar graph representing (Tumor Treatment Model) reduced hepatic tumor number in response to control (saline), Salmonella typhimurium χ4550 and Salmonella typhimurium χ4550pIL2.

FIG. 6 shows a bar graph representing (Tumor Treatment Model) reduced hepatic tumor volume in response to control (saline), Salmonella typhimurium χ4550 and Salmonella typhimurium χ4550pIL2.

FIG. 7 shows a bar graph representing (Tumor Treatment Model) elevated hepatic NK cells in response to control (saline), Salmonella typhimurium χ4550 and Salmonella typhimurium χ4550pIL2.

FIG. 8 shows a bar graph representing (Tumor Treatment Model) elevated hepatic CD8+ cells in response to control (saline), Salmonella typhimurium χ4550 and Salmonella typhimurium χ4550pIL2.

FIG. 9 shows a bar graph representing (Tumor Treatment Model) reduced tumor number in response to saline, antioxidant oil, Salmonella typhimurium χ4550pIL2 and Salmonella typhimurium χ4550pIL2 plus antioxidant oil.

FIG. 10 shows a bar graph representing (Tumor Treatment Model) reduced tumor volume in response to control (saline), antioxidant oil, Salmonella typhimurium χ4550pIL2 and Salmonella typhimurium χ4550pIL2 plus antioxidant oil.

FIG. 11 shows a graph representing (Tumor Prevention Model) improved long term survival in response to control (saline), Salmonella typhimurium χ4550 and Salmonella typhimurium χ4550pIL2.

FIG. 12 shows a bar graph representing (Tumor Prevention Model) increased NK cell population in response to control (saline), antioxidant oil, Salmonella typhimurium χ4550pIL2 and Salmonella typhimurium χ4550pIL2 plus antioxidant oil.

FIG. 13 shows a bar graph representing (Tumor Prevention Model) CD8+ cell population in response to control (saline), antioxidant oil, Salmonella typhimurium χ4550pIL2 and Salmonella typhimurium χ4550pIL2 plus antioxidant oil.

FIG. 14 shows a bar graph representing (Tumor Prevention Model) CD4+ T helper cell population in response to control (saline), oil, Salmonella typhimurium χ4550 pIL2 and Salmonella typhimurium χ4550pIL2 plus antioxidant oil.

FIG. 15 shows a bar graph representing (Tumor Prevention Model) tumor number in response to control (saline), antioxidant oil, Salmonella typhimurium χ4550pIL2 and Salmonella typhimurium χ4550pIL2 plus antioxidant oil.

FIG. 16 shows a bar graph representing (Tumor Prevention Model) tumor volume in response to control (saline), antioxidant oil, Salmonella typhimurium χ4550pIL2 and Salmonella typhimurium χ4550pIL2 plus antioxidant oil.

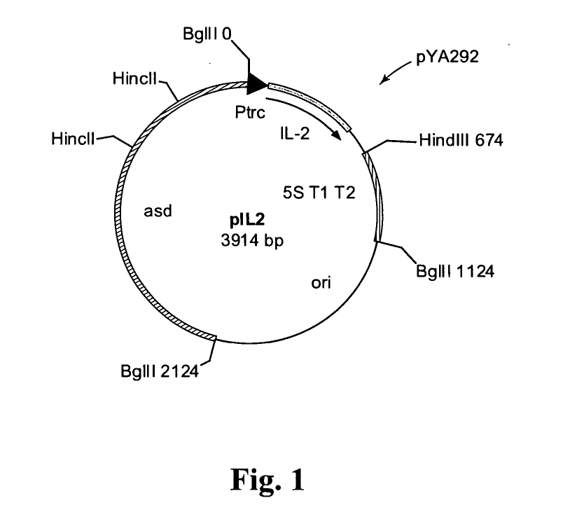

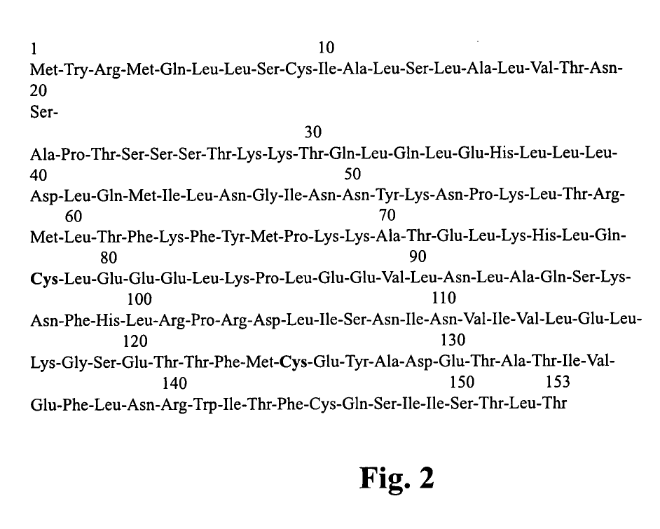

### SUMMARY

The present invention provides a method for treating cancer. The method includes administering to a patient a composition comprising an effective amount of attenuated Salmonella typhimurium containing a plasmid carrying a coding sequence encoding for human interleukin-2. An oil containing a high antioxidant concentration selected from the group consisting of black raspberry oil, red raspberry oil, blackberry oil, marionberry oil, boysenberry oil, evergreen blackberry oil and black cumin oil is also administered as part of the method.

The present invention further provides the plasmid carrying the coding sequence encoding for human interleukin-2 is pYA292.

The present invention also provides the attenuated Salmonella typhimurium containing the coding sequence encoding for human interleukin-2 lacks the cyclic AMP and cAMP receptor protein.

The present invention further provides the attenuated Salmonella typhimurium lacking the enzyme aspartate semialdehyde dehydrogenase and the pYA292 plasmid containing the enzyme aspartate semialdehyde dehydrogenase, which renders the attenuated Salmonella typhimurium harmless and simultaneously expresses the gene for human IL-2.

The present invention also provides the oil containing a high antioxidant concentration being extracted by using a high pressure press and maintaining the temperature of the oil below one hundred twenty degrees Fahrenheit.

The present invention further provides a method wherein the coding sequence encoding human interleukin-2 has an eighty percent identity to SEQ ID NO:2.

The present invention also provides a method wherein the coding sequence encoding human interleukin-2 has an eighty five percent identity to SEQ ID NO:2.

The present invention further provides a method wherein the coding sequence encoding human interleukin-2 has a ninety percent identity to SEQ ID NO:2.

The present invention further provides a method wherein the coding sequence encoding human interleukin-2 has a ninety five percent identity to SEQ ID NO:2.

The present invention further provides a method wherein a dose containing approximately 10⁶ to 10⁸ of the attenuated Salmonella typhimurium containing a plasmid carrying the coding sequence encoding human interleukin-2 is administered once during treatment and a dose of approximately a half teaspoon of an oil containing a high antioxidant concentration selected from the group consisting of black raspberry oil, red raspberry oil, blackberry oil, marionberry oil, boysenberry oil, evergreen blackberry oil and black cumin oil is administered twice a day.

### DETAILED DESCRIPTION

Definitions

“Attenuated” means bacteria selected or altered to greatly diminish its capacity to cause disease, but still able to retain its ability to colonize the gut associated lymphoid tissue.

“CD4+” and “CD4+ cell” mean a helper subset of T cells.

“CD8+” and “CD8+ cell” mean a cytotoxic subset of T cells.

“Coding sequence” and “coding region” are used interchangeably and refer to a polynucleotide that encodes a protein and, when placed under the control of appropriate regulatory sequences, expresses the encoded protein. The boundaries of a coding region are generally determined by a translation start codon at its 5′ end and a translation stop codon at its 3′ end.

“Gated Lymphocytes” refers to lymphocytes that have been analyzed in a fluorescent cell sorter.

“IL-2” means the protein human interleukin-2.

“NK” or “NK cell” means natural killer cell.

“Oil” refers to highly potent antioxidant oils.

“Operably linked” refers to a juxtaposition wherein the components so described are in a relationship permitting them to function in their intended manner. A regulator sequence is operably linked to a coding region when it is joined in such a way that expression of the coding region is achieved under conditions compatible with the regulatory sequence.

“Regulatory Sequence” refers to a nucleotide sequence that regulates expression of a coding region to which it is operably linked. Nonlimiting examples of regulatory sequences include promoters, transcription initiation sites, translation start sites, translation stop sites and terminators.

Human Interleuken-2

Interleukin-2 (IL-2) (SEQ ID NO: 1) is a protein naturally produced by the human body which promotes lymphocyte proliferation and enhances the cytolytic function of T cells and natural killer (NK) cells. It is thus able to stimulate the immune system to produce cancer-destroying white blood cells. IL-2 based immunotherapy in certain types of cancer has been studied for years with limited success.

Attenuated Salmonella typhimurium

While IL-2 is naturally produced by the human body, its maximum effectiveness requires a higher concentration and more specific delivery vector to the disease site. However, high doses of IL-2 are found to result in severe toxicity in many patients. A solution to this problem was found in using a live bacterial strain of Salmonella typhimurium which was attenuated to greatly diminish its capacity to cause disease. S. typhimurium is used due to its natural ability to colonize the gut associated lymphoid tissue (GALT), liver and spleen. Colonization of the liver by the attenuated S. typhimurium further initiates a generalized cellular response against the bacteria or can persist as a carrier state. The χ4550 strain of S. typhimurium used in the present invention is attenuated by using transposon mutagenesis with Tn10 followed by selection for furasic acid resistance. This method of genetic alteration leads to deletional loss of Tn10 and adjacent DNA sequences to produce deletion mutation of aspartate semialdehyde dehydrogenase (asd). These mutations impose a requirement for diaminopimelic acid to render S. typhimurium avirulent without impairing the ability of the bacteria to induce a generalized secretory response. The lack of the asd enzyme in these bacteria leads to the inability to construct a stable cell wall and insure the death of the S. typhimurium. Thus, to insure stable expression of a desired protein, a plasmid (pYA292) was selected which carries the asd gene and the gene was placed using electroporation. Transfecting the plasmid (pYA292) allows for the stable expression of IL-2. In order to insure the avirulence of the S. typhimurium strain, standard auxotrophic techniques using the mouse virulent strain S. typhimurium SR-11 strain χ3306 to construct the χ4550 strain that lacks the ability to synthesize adenylate cyclase and the cAMP receptor protein (CRP), were employed. Cyclic AMP and cAMP receptor protein are necessary for the transcription of many genes and operons concerned with the transport and breakdown of catabolites. Although cAMP is found in mammalian tissue and theoretically could be used by the bacteria to increase the potential for virulence, the lack of a cAMP receptor protein should abolish any benefit that could occur by the uptake of cAMP by these mutant bacteria.

A human cDNA clone for IL-2, optimized for expression in Escherichia coli was cloned into plasmid pYA292 using well known methods and the new transformant renamed χ4550(pIL2). S. typhimurium χ4550pIL2 was constructed from the deletion mutation parent strain by inserting human cDNA for IL-2 into the pYA292 plasmid followed by electroporation. Both the aspartate semialdehyde dehydrogenase (asd+) vector and the human IL-2 clone were digested to completion with restriction enzymes EcoRI (Promega, Madison, Wis.) and Hind III (New England Biolabs, Beverly, Mass.). The ˜3.4 kb linearized vector fragment of pYA292 and the EcoRI-HindIII fragment of the IL-2 gene were isolated following agarose gel electrophoresis using the PrepaGene Kit (BioRad, Hercules, Calif.). The IL-2 gene fragment was ligated into the pYA292 vector using T4 DNA ligase (Promega, Madison, Wis.) with a 3:1 molar excess of insert and incubating for 4 hours at 16° C. The ligation mix was then electroporated into the χ4550 strain of attenuated S. typhimurium.

S. typhimurium, Δcya-1 Δcrp-1 DasdA 1 strain χ4550, was grown in Luria Broth (Sigma, St. Louis, Mo.) containing 50 mg/ml diaminopimelic acid (DAP). Cultures were grown to an absorbance of 0.200 at OD₆₀₀ (approximately 10⁸ colony forming units (CFU)/ml broth) and the cells were prepared for electroporation. Plasmid vector pYA292 and the ligation mix were electroporated into χ4550 utilizing an electroporation device (BioRad) with 0.2 cm disposable cuvettes. Cells were pulsed at 2.5 kV and 25 μF with a pulse controller at 200 ohms. Cells were then subsequently plated on Luria agar without DAP and recombinant clones were identified using the Magic Mini-Prep DNA Purification System (Promega), and restriction enzyme digestion with EcoRI and HindIII and gel electrophoresis with 1.2 agarose. The restriction enzyme mapping revealed a plasmid corresponding to that expected for an insert of the IL-2 fragment in pYA292 and the plasmid was renamed pIL2.

As discussed above, stability of this vector is maintained because the particular strain of S. typhimurium used here (χ4550) lacks the enzyme aspartate semialdehyde dehydrogenase (asd), which, conversely, the plasmid containing the IL-2 gene (pIL2) contains. Bacteria lacking asd cannot make diaminopimelic acid (DAP), an essential component of the bacterial cell wall and, thus, would not long survive. Thus, if the attenuated S. typhimurium were to attempt to revert to its wild-type strain and lose the plasmid, it would die a “DAP-less” death. Because the loss of the IL-2 containing plasmid would also result in the loss of the plasmid encoded asd, stable expression of the IL-2 gene is achieved.

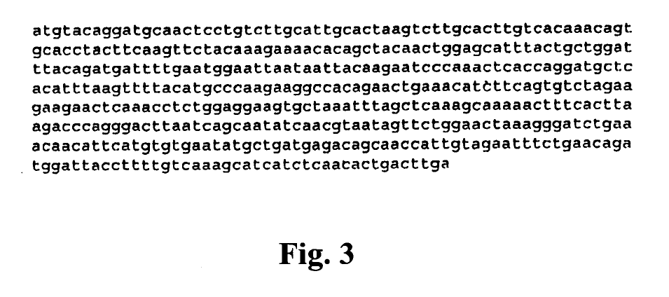

It is believed that a coding sequence encoding human interleukin-2 having an eighty percent identity to SEQ ID NO:2 (as shown in FIG. 3) would be effective in the treating and preventing cancerous tumors. It is further believed that a coding sequence encoding human interleukin-2 having an eighty five percent identity to SEQ ID NO:2 (as shown in FIG. 3) would be effective in treating and preventing cancerous tumors. It is also believed that a coding sequence encoding human interleukin-2 having a ninety percent identity to SEQ ID NO:2 (as shown in FIG. 3) would be effective in treating and preventing cancerous tumors. It is further believed that a coding sequence encoding human interleukin-2 having a ninety five percent identity to SEQ ID NO:2 (as shown in FIG. 3) would be effective in treating and preventing cancerous tumors.

High Antioxidant Plant Oils

Natural oils derived from plant materials are produced through a method of cold pressing the raw plant material. In a preferred embodiment waste products of black raspberries, Rubus occidentalis L. pulp fiber including seeds is recovered and dried to a moisture content of less than 10 percent in a low temperature dryer having rapid air flow and with temperatures not exceeding 120 degrees Fahrenheit. Rapid drying of the seed and pulp material is important to prevent the growth of microorganisms and decay. Following drying, the seeds are separated from the pulp using a seed cleaner.

The seeds are then carefully pressed in a cold press where temperatures of the extracted oil do not exceed 120 degrees Fahrenheit. A Komet™ single or double screw expeller, Model DD85, manufactured by IBG Montforts GmbH was used. Other cold presses are commercially available and could also satisfactorily be used to extract the oil. Typically the press achieves pressures of 1200 pounds per square inch against the seed material and press head. The press may be electrically driven or driven by other mechanical means. The press cylinder, where the oil exits the press, may be enclosed within a hood and put under pressure of an inert gas such as nitrogen or carbon dioxide to prevent the freshly expelled oil from contact with oxygen to enhance the oxidative stability of the oil. To start the process no external heat is used. This is unlike other conventional pressing methods for grains and vegetable oils which add heat to the pressing head. Using a speed of 20 to 100 rpm of the Komet™ press, the oil is extracted at ambient temperatures of 70 to 90 degrees Fahrenheit. A nozzle at the press head having a round aperture ranging from 6 mm to 15 mm allows the solid seed material to be expelled from the press. The oil flows by gravity to a collection container. In one embodiment the fresh, carefully expelled oil is allowed to settle to the bottom of the container and the oil clarifies. In another embodiment the oil is collected and used in an unsettled condition. A filter can also be used for separation of the fine fiber material from the oil. The oil is then decanted for further storage and bottling.

The resulting oil is extremely high in antioxidants, as the following tables show:

TABLE 1
Sterols

| | |
|---|---|
| Cholesterol | <1.0 mg/100 g |
| Campesterol | 26.2 mg/100 g |
| Stigmasterol | 10.2 mg/100 g |
| Beta Sitosterol | 461 mg/100 g |

TABLE 2
Fatty Acid Composition of Black Raspberry Seed Oil

| Fatty Acid | grams/100 g |
|---|---|
| Myristic | nd |
| Palmitic | 1.2-1.6 |
| Palmitoleic | nd |
| Stearic | 0.1 |
| Oleic | 6.1-7.7 |
| Linoleic | 55.8-57.6 |
| Arachidonic | nd |
| Total Sat | 1.2-1.6 |
| Total MUFA | 6.1-7.7 |
| Total ω-3 FA | 35.2 |
| Total PUFA | 91.1-93.0 |

nd = not detectable.
MUFA and PUFA represent mono and polyunsaturated fatty acids.
ω-3 FA = ω-3 fatty acids.

TABLE 3
Vitamin E

| Total Tocotrienol | grams/100 g |
|---|---|
| Alpha Tocotrienol | <.500 mg |
| Beta Tocotrienol | 2.42 mg |
| Delta Tocotrienol | <.500 mg |
| Gamma Tocotrienol | 3.10 mg |

TABLE 4

| Total Tocopherol | grams/100 g |
|---|---|
| Alpha Tocopherol | 1.64 mg |
| Beta Tocopherol | <.500 mg |
| Gamma Tocopherol | 6.05 mg |
| Delta Tocopherol | <.500 mg |

TABLE 5
Antioxidant Property of Black Raspberry Seed Oil

| | |
|---|---|
| ABTS⁺ Scavenging Activity (μmole TE/g) | 0/3-0/7 |
| DPPH Scavenging Activity (% DPPH | 11.4-53.4 |
| quenched | |
| TPC (μg GE/g) | 35.1-92.6 |

ABTS⁺ scavenging activity was evaluated using the radical cation generated by a chemical method.
DPPH scavenging activity was measured at a final concentration of 42 mg oil equivalent/mL or 125 μg meal equivalent/mL in the radical-antioxidant reaction mixture.
TPC = total phenolic content and is expressed as gallic acid equivalent (GE). The TPC was measured using the Folin-Ciocalteu reagent. Values were means of triplicate measurements.

Methods for Tests

Fatty Acid Analysis. One mg of each oil sample was used to prepare the fatty acid methyl esters (FAME) for gas chromatograph (GC) analysis. The GC analysis of the FA composition was performed on a HP® 6890 gas chromatograph equipped with an autosampler, Chemstation and FID (Hewlitt Packard Co., Avondale, Pa.). A fused silica capillary column SP™-2380 (30 m×0.25 mm with a 0.25 μm film thickness) from Supelco (Bellafonte, Pa.) was used with helium as the carrier gas. The following temperature program was used: 165° C. for 20 min followed by a 5°/C.min increase to 185° C., which was then held for 10 min.

Radical Cation ABTS⁺ Scavenging Activity. Radical scavenging capacity of black raspberry antioxidant was evaluated against ABTS⁺ generated by the chemical method. 50 μL of black raspberry antioxidants in 50% acetone was diluted with 450 μL of 7% RMCD to obtain the testing samples. ABTS⁺ was prepared by oxidizing 5 mM aqueous solution of ABTS, 2,2′ -azinobis(3-ethylbenothiazoline-6-sulfonic acid diammonium salt, with manganese dioxide at ambient temperatures for 30 minutes. The ABTS⁺ antioxidant reaction mixture contained 1.0 mL of ABTS⁺ with an absorbance of 0.7 at 734 nm, and 80 μL of 7% RMCD solution for the control. The absorbence at 734 nm was measured at 1 min of the reaction, and the trolox equivalent was calculated using a standard curve prepared with trolox.

Radical DPPH Scavenging Activity. Free radical scavenging capacity of black raspberry oil was determined according to the previously described procedure using the stable 2,2′-diphenyl-1-picryhydrazyl radical (DPPH). The final concentration was 100 μM for DPPH. The absorbance at 517 nm was measured against a blank of pure ethanol at 40 and used to estimate the remaining radical levels according to a standard curve.

Total Phenolic Contents. The total phenolic content of black raspberry seed oil was determined using the Folin-Ciocalteu reagent. In brief, the reaction mixture contained 50 μL of the Folin-Ciocalteu reagent freshly prepared in the laboratory and 0.75 mL of 20% sodium carbonate and 3 mL of pure water. After two hours of reaction at ambient temperature, the absorbance at 765 nm was measured and used to calculate the phenolic contents using gallic acid as a standard.

As indicated above, black raspberry oil has very high concentrations of total vitamin E, tocotrienol, and tocopherols, even higher that other fruit oils from cranberry and grape. Tocotrienols are increasingly being recognized as having an important role in preventing degenerative diseases. Gamma tocopherol, the most potent antioxidant of all the tocopherols is higher in black raspberry oil than in cranberry oil and contributes to oxidative stability of the unsaturated oils in black raspberry oil. The presence of high concentrations of both tocotrienol and tocopherols is unusual as other vegetable oils are more typically high in either tocotrienol or tocopherol but not both.

In another embodiment the oil can be extracted from the raw plant material using Super Critical Fluid Extraction technology. Super Critical Fluid Extraction uses CO₂ under pressure to enter the cell walls of the plant material to force the separation of oils and extracts. By varying temperature and pressure different separations or fractions can be achieved. Because the process works at low temperature and in the absence of oxygen, the resulting oil is of high quality and unaltered from its natural form.

While black raspberry oil appears to be the most promising, it should be mentioned that other natural oils such as those derived from black cumin seed, caneberries (raspberry, blackberry, marionberry, boysenberry and evergreen blackberry), coriander, sea buckthorn, palm fruit oil and cardomon are also known to be high in antioxidants. Finally, combinations of the various oils discussed above are also contemplated by the invention and are therefore within its scope.

Experimental Procedure

Two basic tumor models were used to examine the efficacy of this novel anti-tumor system: a Tumor Treatment Model and a Tumor Prevention Model.

In the Tumor Treatment Model female 6 to 8 week old C57BL/6 mice were purchased from Harlan Sprague-Dawley (Indianapolis, Ind.). At the onset of each experiment, the mice were randomly divided into four groups (Control, Salmonella typhimurium-IL-2, antioxidant oil alone and Salmonella typhimurium IL-2 with antioxidant oil) that were orally inoculated with saline or 10⁸ S. typhimurium χ4550pIL2 and received a standard rodent diet or a standard rodent diet supplemented with antioxidant oil for the duration of the experiment. The procedure yielded four groups: saline, antioxidant oil, S. typhimurium χ4550pIL2, and S. typhimurium χ4550pIL2+antioxidant oil. Mice in the antioxidant oil and S. typhimurium χ4550pIL2+antioxidant oil groups received a standard rodent diet supplemented with black raspberry seed oil from Botanic Oil Innovation, Inc. (Spooner, Wis.) at a concentration of ten percent by weight. In order to incorporate the antioxidant oil into the diet, it was necessary to crush the standard rodent diet pellets to the consistency of coarse sand. To negate any possible variation in food consumption due to the form of the diet, all groups received a crushed diet. Mice were fed their respective diets and water ad libitum. 25,000-100,000 MCA murine adenocarcinoma cells were injected into the spleen to facilitate hepatic metastases via the portal circulation on Day 0. On Day 3 the mice were randomized into their groups and treated. On Day 12 of experimentation, mice were sacrificed and liver metastases were enumerated for number and volume of tumor.

In the Tumor Prevention Model, at the onset of each experiment, mice were administered the control (saline), antioxidant oil, S. typhimurium χ4550pIL2 with and without the antioxidant oil on Day 0. On Day 7 splenic injection of 50,000 MCA-38 adenocarcinoma cells was accomplished. On Day 14 hepatic metastases were enumerated for tumor number and volume. Total tumor volume was calculated assuming tumor shape as a sphere (4/3 r³). Hepatic lymphocytes were also analyzed from each experimental.

Experiments were concluded at 3, 7 or 14 days following oral inoculation and all mice were sacrificed under anesthesia. A splenectomy was performed to allow for splenic lymphocyte analysis. Splenic lymphocytes were prepared by modifying a technique used to isolate hepatic lymphocytes. Briefly, the spleen was mechanically minced, passed through 100-gauge nylon mesh (Sefar America, Inc., Kansas City, Mo.), and suspended in DMEM (Sigma, St. Louis, Mo.) with 10% fetal goat serum (Sigma). Individual specimens were place on lymphocyte separation medium (Mediatech, Inc., Herndon, Va.) and centrifuged at 300 g for 60 minutes at room temperature. The mononuclear cell layer was harvested and washed twice in phosphate buffered solution (Gibco, Grand Island, N.Y.) with centrifugation at 300 g for 10 minutes at room temperature.

Splenic lymphocytes were stained with a combination of fluorochrome-conjugated anti-mouse monoclonal antibodies, including anti-NK1.1, anti-CD4, and anti-CD8 (all obtained from BD Biosciences Pharmingen, San Diego, Calif.). Lymphocyte staining was performed at 4° C. for 30 minutes by incubating the cells with monoclonal antibodies. After washing, analysis was performed with a FACScan cytofluorometer (Becton-Dickinson, Grenoble, France) using CellQuest software (Becton-Dickinson). Viable lymphocytes were gated by side and forward scatter profiles. For each specimen, analysis was based on 10,000 acquired events.

Statistical analyses were performed using StatView 5.0 (SAS Institute, Cary, N.C.). At the conclusion of an experiment, splenic lymphocyte phenotype was analyzed by analysis of variance followed by Fisher's test for significant difference. Experiments were repeated twice on separate days to verify reproducibility. Statistical significance was regarded as P<0.05.

Examples (Tumor Treatment Model)

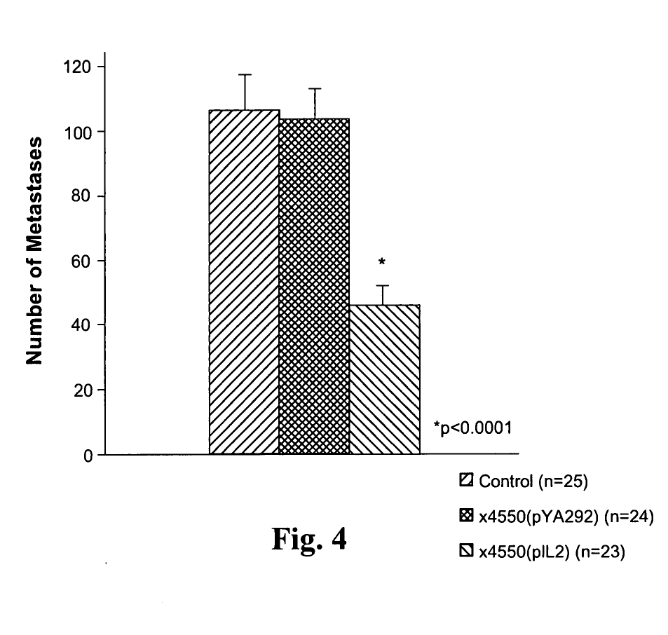

FIG. 4 shows the results of the control, S. typhimurium χ4550 and S. typhimurium χ4550pIL2 groups on hepatic metastases when administered orally to tumor burdened mice. A statistically significant decrease in hepatic colorectal metastases is shown.

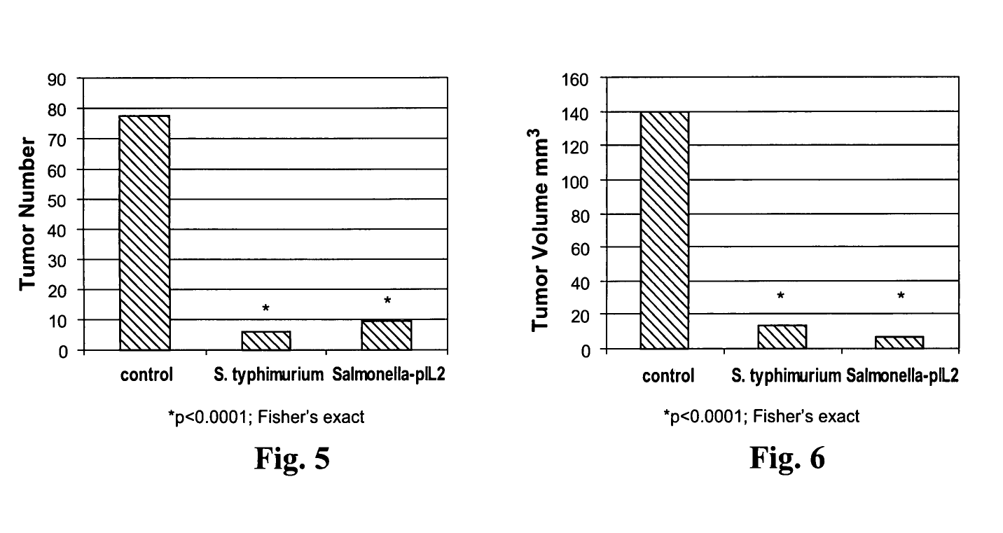

FIG. 5 shows the results of the control, S. typhimurium χ4550 and S. typhimurium χ4550pIL2 groups on tumor number when administered orally to tumor burdened mice. A statistically significant reduction in tumor number is shown.

FIG. 6 shows the results of the control, S. typhimurium χ4550 and S. typhimurium χ4550pIL2 groups on tumor volume when administered orally to tumor burdened mice. A statistically significant reduction in tumor volume is shown.

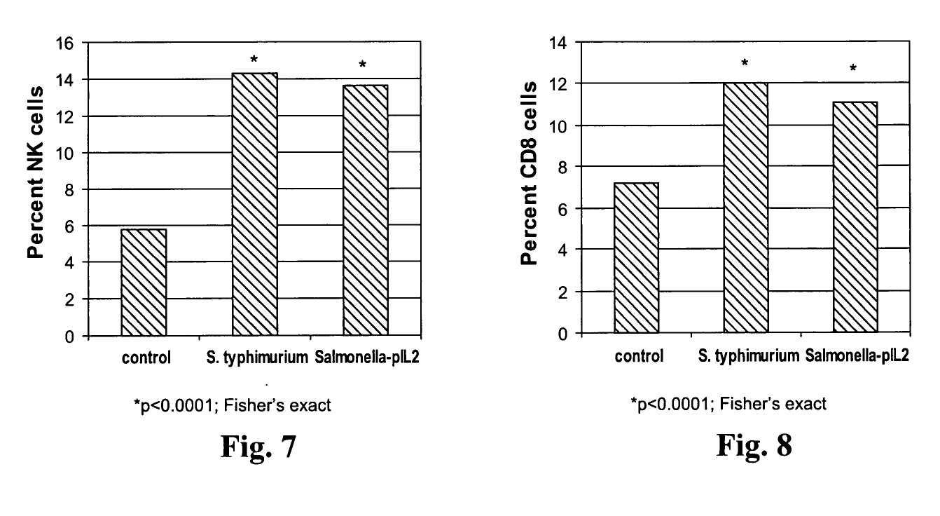

FIG. 7 shows the results of the control, S. typhimurium χ4550 and S. typhimurium χ4550pIL2 groups on NK cells when administered orally to tumor burdened mice. An increase in NK cells is shown in the S. typhimurium χ4550 and S. typhimurium χ4550pIL2 groups when compared to the control group.

FIG. 8 shows the results of the control, S. typhimurium χ4550 and S. typhimurium χ4550pIL2 groups on CD8+ cells when administered orally to tumor burdened mice. An increase in CD8+ cells is shown in the S. typhimurium χ4550 and S. typhimurium χ4550pIL2 groups when compared to the control group.

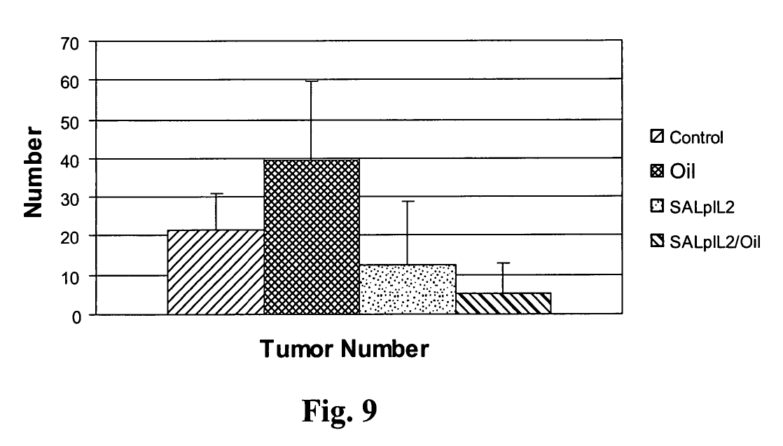

FIG. 9 shows the results of the four groups (control, oil, S. typhimurium χ4550pIL2 and S. typhimurium χ4550pIL2 plus antioxidant oil on total tumor number in tumor burdened mice. As shown, the S. typhimurium χ4550pIL2 plus antioxidant oil group had approximately an eight fold reduction in total number of tumor cells compared to the group receiving only oil. An approximate two fold reduction in the number of tumors was shown compared to the group receiving only S. typhimurium χ4550pIL2. Compared to the control group, the group receiving S. typhimurium χ4550pIL2 plus antioxidant oil showed a four fold reduction in the number of tumors.

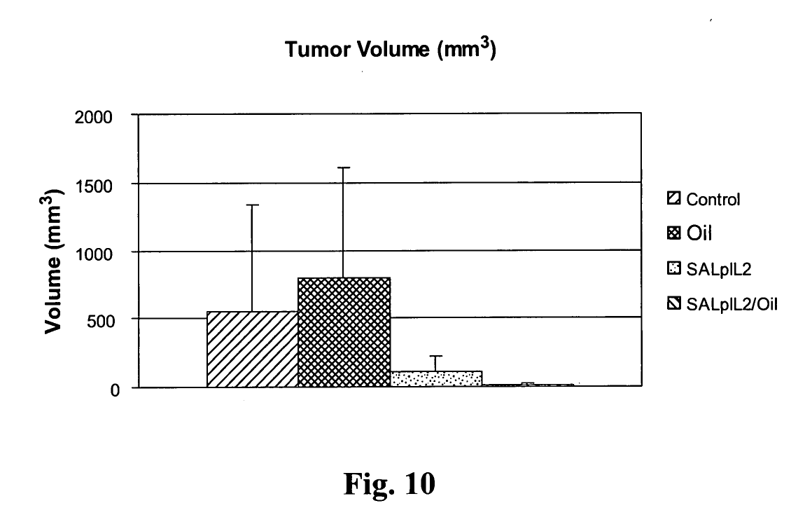

FIG. 10 shows the results of the four groups (control, antioxidant oil, S. typhimurium χ4550pIL2 and S. typhimurium χ4550pIL2+antioxidant oil) on tumor volume in tumor burdened mice. As shown the S. typhimurium χ4550pIL2 plus antioxidant oil group showed almost negligible tumor volume compared to the antioxidant oil only group, the control group, and the S. typhimurium χ4550pIL2 only group.

### EXAMPLES

### Tumor Prevention Model

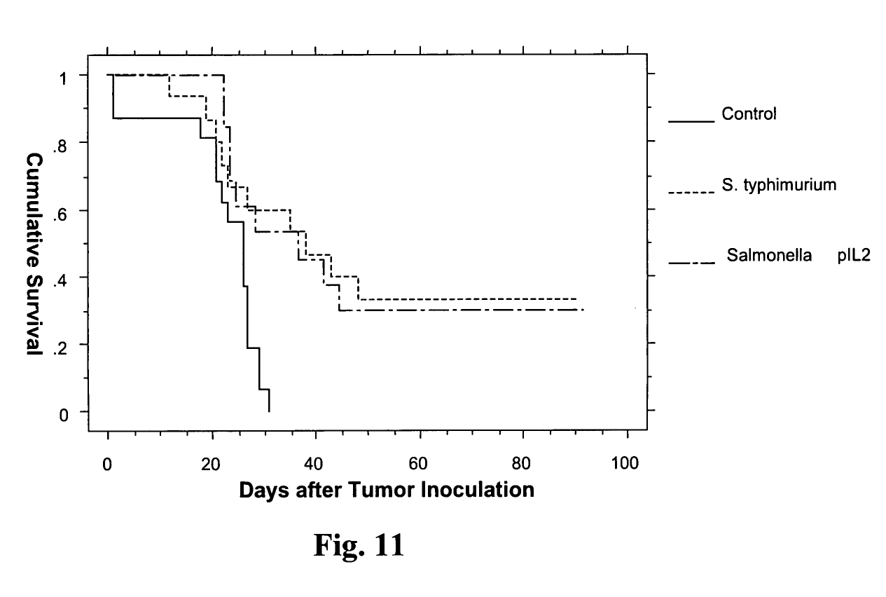

FIG. 11 shows the cumulative survival of tumor naive mice after being fed control, S. typhimurium χ4550 and S. typhimurium χ4550pIL2. The groups receiving S. typhimurium χ4550 and S. typhimurium χ4550pIL2 show an almost 40% long term survival rate over the control group.

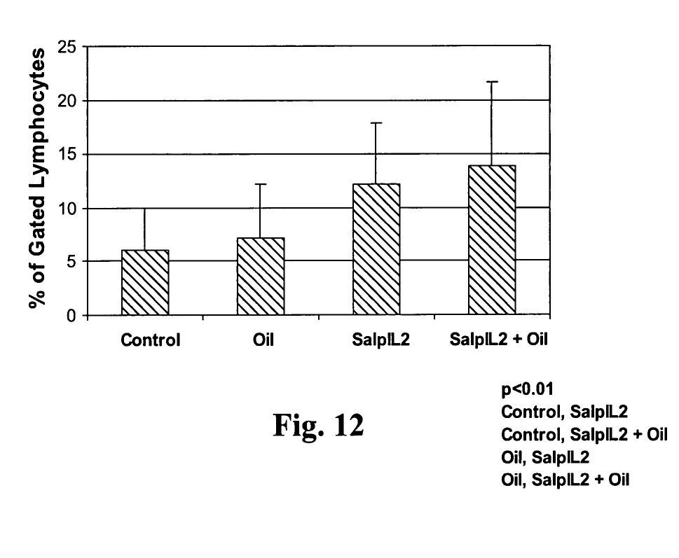

FIG. 12 shows the results of the four groups (control, antioxidant oil, S. typhimurium χ4550pIL2 and S. typhimurium χ4550pIL2 plus antioxidant oil) on NK cell population in tumor naïve mice. A statistically significant increase over the control group and antioxidant oil only group is shown in the S. typhimurium χ4550pIL2 and S. typhimurium χ4550pIL2 plus antioxidant oil groups.

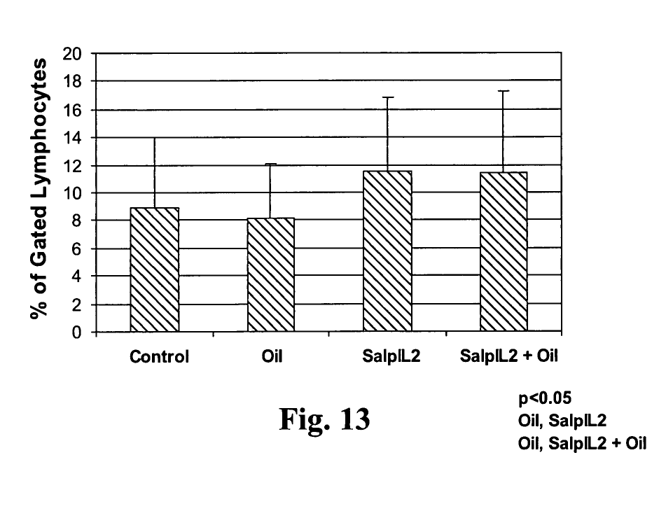

FIG. 13 shows the results of the four groups (control, antioxidant oil, S. typhimurium χ4550pIL2 and S. typhimurium χ4550pIL2 plus antioxidant oil) on CD8+ cell population in tumor naïve mice. A slight increase in CD8+ T cell population is shown in the mice in the S. typhimurium χ4550pIL2 plus antioxidant oil group.

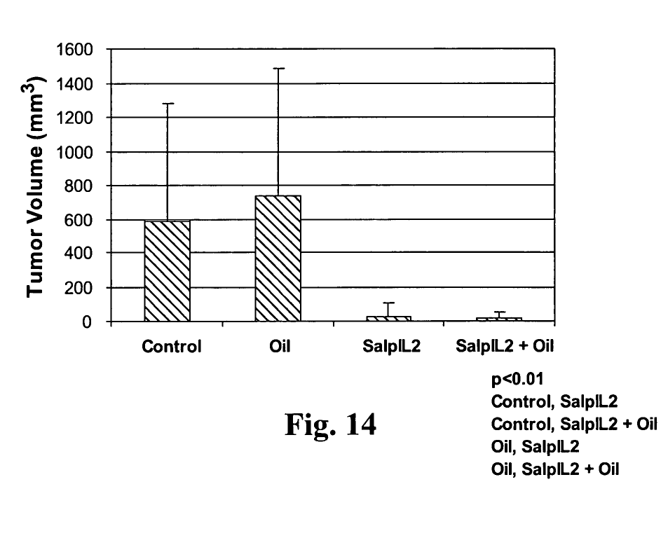

FIG. 14 shows the results of the four groups (control, antioxidant oil, S. typhimurium χ4550pIL2 and S. typhimurium χ4550pIL2 plus antioxidant oil) on CD4+ T helper cell population in tumor naïve mice. An overall statistically significant increase in CD4+ T helper cell population is shown in the S. typhimurium χ4550pIL2 plus antioxidant oil group.

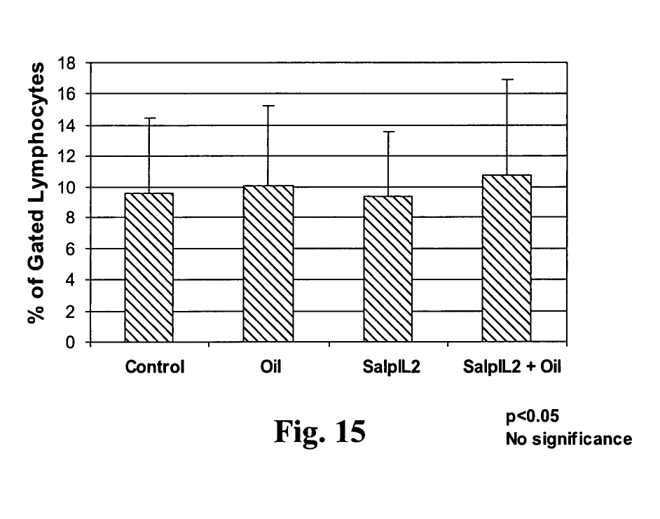

FIG. 15 shows the results of the four groups (control, antioxidant oil, S. typhimurium χ4550pIL2 and S. typhimurium χ4550pIL2 plus antioxidant oil) on tumor number in tumor naïve mice. A statistically significant decrease in tumor number is shown in the S. typhimurium χ4550pIL2 and S. typhimurium χ4550pIL2 plus antioxidant oil groups.

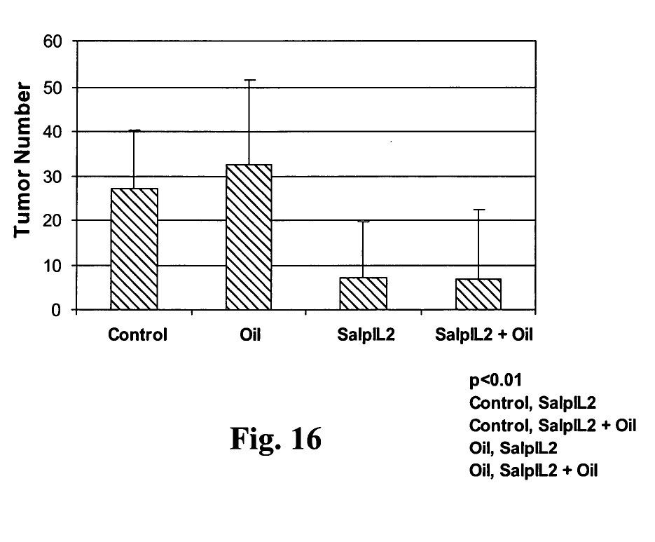

FIG. 16 shows the results of the four groups (control, antioxidant oil, S. typhimurium χ4550pIL2 and S. typhimurium χ4550pIL2 plus antioxidant oil) on tumor volume in tumor naïve mice. A statistically significant decrease in tumor volume is observed in the S. typhimurium χ4550pIL2 group, with a further decrease observed in the S. typhimurium χ4550pIL2 antioxidant oil group.

Proposed Theory/Mechanism

The exact mechanism responsible for the improved colorectal cancer response resulting from the administration of S. typhimurium χ4550pIL2 concurrently with highly potent antioxidant oils is not yet known. We have shown that S. typhimurium χ4550pIL2 alone increases the populations of host effectors cells (NK cells and CD8+ Cytotoxic T cells. Further, it has long been known that both NK cells and CD8⁺ lymphocytes play an important role in the destruction of tumor cells both in vitro and in vivo.

Oxidative stress is thought to play an important role in the pathogenesis of numerous chronic diseases, such as coronary heart disease and cancer. Although there are many factors in the development of theses diseases, considerable experimental evidence has linked the production of free radicals to biological damage that can provide a basis for the initiation and progression of certain diseases. Free radicals are atoms or molecules that are highly reactive with other cellular structures due to an unpaired electron. Consequently, they are capable of chemically altering nearly all major classes of biomolecules (e.g., lipids, nucleic acids and proteins). Free radicals such as the superoxide anion, the hydroxyl radical and singlet oxygen can be produced in vivo by factors such as dietary imbalances, tobacco smoke, pollutants or from sources such as lipid peroxidation, inflammation and biochemical reactions. They are capable of damaging DNA, inhibiting its repair and amplifying viral and oncogene expression. Interestingly, free radicals are also generated by cells of the immune system to destroy invading organisms. However, the presence of these strong oxidants places additional stress on the immune system, which can result in a diminished response against invaders. Furthermore, the long-term presence of these oxidizing species will eventually be detrimental to the human body.

Humans and other aerobic organisms have evolved a variety of mechanisms to protect themselves from the deleterious effects of free radicals. These defense mechanisms protect against free radical damage either directly or indirectly. The defense mechanisms include enzymes such as catalase and superoxide dismutases and repair enzymes such as DNA glycosylases. Water and lipid-soluble antioxidants such as ascorbate (vitamin C), \acute{α}-tocopherol (vitamin E) and beta carotene also act to eliminate free radicals. These antioxidants help shield DNA from the deleterious effects of oxidative damage by absorbing unstable oxygen molecules.

A number of sources indicate a relationship between diet and cancer incidence in humans. The geographic distribution of types of cancer, the changing cancer patters and data from experimental animal studies all indicate that diet and nutrition are important factors in the control and prevention of human cancers. The potential importance of diet in cancer prevention is also noted by the suspected causes of some cancers. Most cancers (an estimated 80% to 90%) have environmental causes and are therefore potentially preventable. Much controversy surrounds the actual percent of cancers associated with dietary factors, but it has been estimated that in men 30% to 40% of all cancers are in some way related to diet. In women, it is believed that 60% of all cancers are related to diet. Another study estimates that 35% of cancer is diet related. Regardless of the exact numbers, these are impressive percentages.

Presumably, pathology due to oxidative stress results when the generation of free radicals exceeds the cell's capacity to protect or repair itself. Therefore, if oxidative damage is an important etiologic factor in the pathogenesis of diseases such as cancer, then it follows that antioxidants, which act to reduce oxidative stress, may play a role in the prevention or treatment of these diseases. The accumulation and growth of free radicals in tissues is often found in association with suppressed immune function, infections such as HPV and HIV, cancer and heart disease. In fact damage to heart blood vessels and the incidence of coronary heart disease has been shown to be reduced with increased dietary antioxidant intake. The protective effects of topical antioxidants (vitamin A derivatives such as retinoic acids) against proliferative dermatological diseases as well as photo-aging have been well documented. Many studies continue to demonstrate below normal antioxidant tissue and blood plasma levels in women with HPV and other cervical neoplasms, while high levels provide protection against their initiation and progression.

Like the B vitamins, the beneficial effects of antioxidants are most notable when combined with one another. In fact, diets high in antioxidants (e.g., the traditional Greek Mediterranean diet) have been shown to be protective against cancer and various diseases. It is believed that antioxidants alter cancer incidence and growth by acting as anticarcinogens. Nutritional anticarcinogens function by (a) inhibition of tumor initiation via alteration of cellular metabolism, (b) picking up active forms of carcinogens and preventing them from reaching target sites, (c) alteration of the body's defense systems, (d) inhibition of cancer progression once it has been initiated by the alteration of cell differentiation, and (e) prevention of gene activation and cellular proliferation by tumor promoters.

It is also known that in individuals, and in entire populations, showing the lowest rates of colorectal cancer occur when diets rich in fruits and vegetables are consumed. One possible mechanism for this phenomenon is that fruits and vegetables contain anticarcinogens, such as antioxidants, that prevent the development of colorectal tumors. It is believed that antioxidants may inhibit the process of lipid peroxidation and reduce the formation of mutagenic peroxidation products in the colon. It is hypothesized that specific receptor populations for NK cells and CD8+ Cytotoxic T cells are increased by the administration of an effective amount of S. typhimurium χ4550pIL2 together with highly potent antioxidant oils, such as black raspberry oil. An alternative hypothesis suggests that the administration of an effective amount of S. typhimurium χ4550pIL2 together with highly potent antioxidant oils, such as black raspberry oil somehow sends a signal to the cellular genetic machinery to increase the number of receptors and/or cell populations themselves.

It is believed that the administration of S. typhimurium χ4550pIL2 concurrently with highly potent antioxidant oils will also be effective in treating infectious disease, other cancerous tumors, slowing the ageing process and treating other immuno-compromised states, including HIV infections. An additional use of the present invention would be administering it to cancer patients between periods of chemotherapy and/or radiation treatments. It is further believed that the composition containing S. typhimurium χ4550pIL2 concurrently with highly potent antioxidant oils will also be effective as a preventive treatment when used in patients with high risk factors for colon cancer such as family or behavioral history.

Use

Prior to the oral delivery of the S. typhimurium χ4550pIL2 and anti-oxidant oil alkalization of the patient's stomach is necessary to neutralize gastric acid to prevent the acid induced destruction of the S. typhimurium χ4550pIL2. This is accomplished by orally administering 30 ml of Bicitra® 15 minutes prior to administering the S. typhimurium χ4550pIL2 with anti-oxidant oil. A dose containing approximately 10⁶ to 10⁸ S. typhimurium χ4550pIL2 is administered to a human patient once, a the initiation of treatment. Approximately one-half teaspoon of cold pressed black raspberry oil is administered to the patient twice a day, throughout the treatment period. As treatment continues, the dosage of each may be escalated or altered as indicated.

While the above description of use of the present invention is specifically directed to human beings, it is also speculated that the present invention would also be effective in treating agricultural (e.g., cattle, swine, sheep, horses, domesticated fowl) and companion (dogs, cats, birds) animals.

Although the present invention has been described in considerable detail with reference to certain preferred versions thereof, other versions are also possible. Therefore, the spirit and scope of the appended claims should not be limited to the description of the preferred versions contained herein.

1. A method for treating cancer, the method comprising administering to a patient a composition comprising an effective amount of attenuated Salmonella typhimurium containing a plasmid carrying a coding sequence encoding human interleukin-2 and an oil containing a high antioxidant concentration selected from the group consisting of black raspberry oil, red raspberry oil, blackberry oil, marionberry oil, boysenberry oil, evergreen blackberry oil and black cumin oil:

2. The method of claim 1 wherein the plasmid carrying the coding sequence encoding for human interleukin-2 is pYA292.

3. The method of claim 1 wherein the attenuated Salmonella typhimurium containing the coding sequence encoding human interleukin-2 lacks the cyclic AMP and cAMP receptor protein.

4. The method of claim 1 wherein the attenuated Salmonella typhimurium lacks the enzyme aspartate semialdehyde dehydrogenase and the pYA292 plasmid contains the enzyme aspartate semialdehyde dehydrogenase, which renders the attenuated Salmonella typhimurium harmless and simultaneously codes for human IL-2.

5. The method of claim 1 wherein the oil containing a high antioxidant concentration is extracted by using a high pressure press and maintaining the temperature of the oil below one hundred twenty degrees Fahrenheit.

6. The method of claim 1 wherein the coding sequence encoding human interleukin-2 has an eighty percent identity to SEQ ID NO:2.

7. The method of claim 1 wherein the coding sequence encoding human interleukin-2 has an eighty five percent identity to SEQ ID NO:2.

8. The method of claim 1 wherein the coding sequence encoding human interleukin-2 has a ninety percent identity to SEQ ID NO:2.

9. The method of claim 1 wherein the coding sequence encoding human interleukin-2 has a ninety five percent identity to SEQ ID NO:2.

10. The method of claim 1 wherein a dose containing approximately 10⁶ to 10⁸ of the attenuated Salmonella typhimurium containing a plasmid carrying the coding sequence encoding human interleukin-2 is administered once during treatment and a dose of approximately a half teaspoon of the oil containing a high antioxidant concentration selected from the group consisting of black raspberry oil, red raspberry oil, blackberry oil, marionberry oil, boysenberry oil, evergreen blackberry oil and black cumin oil is administered twice a day.

11. A method for treating cancer and simultaneously boosting a patient's immune system comprising administering to the patient an oil containing a high antioxidant concentration selected from the group consisting of black raspberry oil, red raspberry oil, blackberry oil, marionberry oil, boysenberry oil, evergreen blackberry oil and black cumin oil.

12. The method of claim 11 wherein the patient is administered a dose of approximately one half teaspoon of the oil having a high antioxidant concentration twice per day.

13. The method of claim 11 wherein the oil containing a high antioxidant concentration is extracted by using a high pressure press and maintaining the temperature of the oil below one hundred twenty degrees Fahrenheit.

14. A method for treating cancer comprising administering to a patient a composition comprising an initial combined dose of an effective amount of attenuated Salmonella typhimurium containing a plasmid carrying a coding sequence encoding human interleukin-2 and an oil containing a high antioxidant concentration selected from the group consisting of black raspberry oil, red raspberry oil, blackberry oil, marionberry oil, boysenberry oil, evergreen blackberry oil and black cumin oil, followed by daily administration of the oil containing a high antioxidant concentration selected from the group consisting of black raspberry oil, red raspberry oil, blackberry oil, marionberry oil, boysenberry oil, evergreen blackberry oil and black cumin oil.

15. The method of claim 14 wherein the plasmid carrying the coding sequence encoding for human interleukin-2 is pYA292.

16. The method of claim 14 wherein the attenuated Salmonella typhimurium containing the coding sequence encoding human interleukin-2 lacks the cyclic AMP and cAMP receptor protein.

17. The method of claim 14 wherein the attenuated Salmonella typhimurium lacks the enzyme aspartate semialdehyde dehydrogenase and the pYA292 plasmid contains the enzyme aspartate semialdehyde dehydrogenase, which renders the attenuated Salmonella typhimurium harmless and simultaneously codes for human IL-2.

18. The method of claim 14 wherein the oil containing a high antioxidant concentration is extracted by using a high pressure press and maintaining the temperature of the oil below one hundred twenty degrees Fahrenheit.

19. The method of claim 14 wherein the daily dose of the oil having a high antioxidant concentration is approximately one half teaspoon twice per day.

20. The method of claim 14 wherein the coding sequence encoding human interleukin-2 has an eighty percent identity to SEQ ID NO:2.

21. The method of claim 14 wherein the coding sequence encoding human interleukin-2 has an eighty five percent identity to SEQ ID NO:2.

22. The method of claim 14 wherein the coding sequence encoding human interleukin-2 has a ninety percent identity to SEQ ID NO:2.

23. The method of claim 14 wherein the coding sequence encoding human interleukin-2 has a ninety five percent identity to SEQ ID NO:2.

24. A composition for treating cancer, comprising an effective amount of attenuated Salmonella typhimurium containing a plasmid carrying a coding sequence encoding human interleukin-2 and an oil containing a high antioxidant concentration selected from the group consisting of black raspberry oil, red raspberry oil, blackberry oil, marionberry oil, boysenberry oil, evergreen blackberry oil and black cumin oil.

25. The composition of claim 24 wherein the plasmid carrying the coding sequence encoding for human interleukin-2 is pYA292.

26. The composition of claim 24 wherein the attenuated Salmonella typhimurium containing the coding sequence encoding human interleukin-2 lacks the cyclic AMP and cAMP receptor protein.

27. The composition of claim 24 wherein the attenuated Salmonella typhimurium lacks the enzyme aspartate semialdehyde dehydrogenase and the pYA292 plasmid contains the enzyme aspartate semialdehyde dehydrogenase, which renders the attenuated Salmonella typhimurium harmless and simultaneously codes for human IL-2.

28. The composition of claim 24 wherein the oil containing a high antioxidant concentration is extracted by using a high pressure press and maintaining the temperature of the oil below one hundred twenty degrees Fahrenheit.

29. The composition of claim 24 wherein the coding sequence encoding human interleukin-2 has an eighty percent identity to SEQ ID NO:2.

30. The composition of claim 24 wherein the coding sequence encoding human interleukin-2 has an eighty five percent identity to SEQ ID NO:2.

31. The composition of claim 24 wherein the coding sequence encoding human interleukin-2 has a ninety percent identity to SEQ ID NO:2.

32. The composition of claim 24 wherein the coding sequence encoding human interleukin-2 has a ninety five percent identity to SEQ ID NO:2.
